(19) United States
(12) Patent Application Publication
(10) Pub. No.: US 2021/0169423 A1
(43) Pub. Date: Jun. 10, 2021

(54) Device for correcting position of heart when measuring blood pressure

(71) Applicant: Yuanhong MAO, Nantong (CN)

(72) Inventors: Yuanhong MAO, Nantong (CN); Shenchu GONG, Nantong (CN); Hong YANG, Nantong (CN); Juhua LU, Nantong (CN); Aihong YUAN, Nantong (CN); Shu ZHOU, Nantong (CN)

(21) Appl. No.: 16/771,238

(22) PCT Filed: Nov. 1, 2018
(86) PCT No.: PCT/CN2018/113296

§ 371 Date: Jun. 10, 2020

(30) Foreign Application Priority Data
Oct. 26, 2018 (CN) 201811258737.8

(51) Int. Cl. A61B 5/00; C09D 7/61; C09D 163/00; A61G 15/02

(52) U.S. Cl. CPC A61B 5/704; C09D 7/61; A61B 5/021; A61G 15/02; C09D 163/00

(57) ABSTRACT

The invention discloses a device for correcting the position of the heart when measuring blood pressure, which comprises a base, and a first support unit, a second support unit and a third support unit are arranged in parallel on the upper part of the base. The present invention adopts an integrated design, which makes the structure of the whole device relatively stable, and can avoid accidents that medical staff and patients fall when measuring blood pressure, especially for hypertensive patients over 75 years old. It directly threatens its life safety, thereby effectively ensuring the safety of the patient at the time of the visit; it can proofread the patient's heart position when measuring blood pressure, which can ensure that the patient's right arm elbow is on the same level as the patient's heart, thereby making the patient

### TECHNOSPHERE

The invention relates to the technical field of blood pressure measurement, and more specifically, it relates to a device for correcting the position of the heart when measuring blood pressure.

### BACKGROUND TECHNOLOGY

Blood pressure refers to the lateral pressure that acts on the blood vessel wall per unit area when blood flows in a blood vessel. It is called arterial blood pressure, capillary pressure and venous blood pressure in different blood vessels. The blood pressure is usually referred to as the arterial blood pressure of the systemic circulation.

Hypertension is characterized by an increase in systemic arterial blood pressure (systolic and/or diastolic blood pressure) (systolic blood pressure ≥140 mm Hg, diastolic blood pressure ≥90 mm Hg), which may be accompanied by the functions of organs such as heart, brain, kidney Or clinical syndrome of organic damage. Hypertension is the most common chronic disease and the most important risk factor for cardiovascular and cerebrovascular diseases. Normal people's blood pressure fluctuates within a certain range with changes in the internal and external environment. In the overall population, blood pressure levels gradually increased with age, and systolic blood pressure was more obvious, but diastolic blood pressure showed a downward trend after 50 years of age, and pulse pressure also increased.

Blood pressure measurement is one of the must-do items for patients with hypertension. However, there are the following problems in the process of measuring blood pressure in patients with hypertension: When patients measure blood pressure, patients will sit in chairs prepared by the hospital The arm is placed on the diagnosis and treatment table. The height of the diagnosis table and chair is fixed, and the height will not be adjusted due to the difference in patient height. Patients of different heights use the same height table and chair; in this way, the patient is measuring blood pressure At this time, it is inevitable that the brachial artery of the patient's upper arm and the patient's heart are on the same level. If the arm for measuring blood pressure is too high or too low, the blood pressure measurement value will be inaccurate, which will make the patient's blood pressure measurement result inaccurate, which is not conducive to the patient's subsequent treatment, especially to the doctor's control of the patient's medication. Because the blood pressure measured by the individual patient is inaccurate, it cannot provide the patient with a reliable basis for medication, which greatly affects the control of blood pressure in patients with hypertension, resulting in cardiovascular and cerebrovascular accidents, leading to patient disability and even death. Therefore, a device for correcting the position of the heart when measuring blood pressure is proposed to solve the above problem.

### CONTENTS OF THE INVENTION

In view of the shortcomings of the prior art, the object of the present invention is to provide a device for correcting the position of the heart when measuring blood pressure, which adopts an integrated design, which makes the structure of the entire device relatively stable and can effectively prevent the first support unit and the second The falling phenomenon of the support unit and the third support unit can avoid accidents that medical staff and patients fall when measuring blood pressure, especially for hypertensive patients over 75 years old, accidental falls directly threaten their lives Safe, so as to effectively ensure the safety of the patient at the time of visit; secondly, it can proofread the patient's heart position when measuring blood pressure, which can ensure that the patient's right arm elbow is on the same level as the patient's heart, so that the patient's blood pressure The measurement results are more accurate and the error is smaller, which is conducive to the subsequent treatment of the patient, especially for the doctor to control the patient's medication; in addition, it has good practicality, so that the fatter patients can easily sit on the third support The upper part of the board; in addition, the protective layer prepared by it can effectively increase the anti-corrosion, abrasion resistance, anti-skid and anti-aging properties of the base, and can prevent medical staff and patients from slipping when using the device.

To achieve the above objectives, the present invention provides the following technical solutions:

A device for correcting the position of the heart when measuring blood pressure, including a base, and a first support unit, a second support unit, and a third support unit are sequentially arranged in parallel on the upper part of the base;

The first support unit includes four first support rods and first support plates, the four first support rods are symmetrically and vertically fixedly mounted on the upper portion of the base, and the first support plates are fixedly installed on four The upper end of the first support rod;

The second support unit includes four second support rods and a second support plate, the four second support rods are symmetrically and vertically fixedly mounted on the upper portion of the base, and the second support plates are fixedly mounted on four The upper end of the second support rod;

The third support unit includes a cylindrical casing fixedly mounted on the upper portion of the base, the upper end of the cylindrical casing is an open structure, and the upper portion of the inner bottom wall of the cylindrical casing is centrally fixedly installed There is a motor, the rotating shaft of the motor is fixedly installed with a screw through a coupling, the inside of the cylindrical housing is located above the motor, and a cylindrical support block is slidably installed, and the cylindrical housing The upper part of the inner bottom wall of the body is symmetrically fixed around the motor, and two vertically arranged guide rods are symmetrically fixed. The upper ends of the two guide rods slide into the bottom of the cylindrical support block. The upper end of the rod is screwed inside the bottom of the cylindrical support block, and a circular plate is fixedly installed on the upper portion of the cylindrical support block, the circular plate is located above the cylindrical housing, and the circular plate A third support plate is fixedly installed on the upper part of the rotating member.

By adopting the above technical solution, the first support unit is used as a seat when medical staff diagnose the patient; the second support unit is used as an examination table when measuring blood pressure; the third support unit is used as a seat when the patient measures blood pressure; the device The first support unit, the second support unit and the third support unit are all arranged on the upper part of the base, and the integrated design is adopted, so that the structure of the whole device is relatively stable, which can effectively prevent the first support unit, the second support unit and the third support The unit's dumping phenomenon can avoid accidents that medical staff and patients fall when measuring blood pressure, especially for hypertensive patients over 75 years old, accidental falls directly threaten their lives and safety. In addition, the third The support unit is driven by a motor to adjust the height of the third support plate, so when the patient is sitting on the upper part of the third support plate, the position of the patient can be adjusted by controlling the motor, while the patient puts his right arm on the second support plate The upper part of the patient can be adjusted so that the elbow of the patient's right arm is on the same level as the patient's heart. At this time, the patient's heart position when the blood pressure is measured is corrected, so that the patient's blood pressure is measured The results are more accurate and the error is smaller, which is conducive to the follow-up treatment of the patient, especially for the doctor to control the patient's medication.

Further, a plurality of rubber feet are fixedly installed on the bottom of the base.

By adopting the above technical solution, several rubber feet have better support for the entire device when the device is placed, at the same time, it can prevent the base from scratching the support surface of the device and increase the support surface between the base and the device The static friction force can prevent the device from slipping during use.

Further, a protective baffle is fixedly installed on the upper part of the third support plate.

By adopting the above technical solution, the protective baffle is used to protect the patient when the patient is sitting on the upper part of the third support plate, which can prevent the patient from accidentally falling from the upper part of the third support plate, thereby making the device safe and reliable.

Further, the rotating member includes an annular outer ring and an annular inner ring, an inner bottom of the annular outer ring is integrally provided with a retaining ring, an upper portion of the annular inner ring is integrally provided with a limit ring, and the annular inner ring passes through The limit ring and the retaining ring are movably engaged inside the annular outer ring, and the bottom of the annular inner ring is fixedly connected to the upper part of the circular plate, and the upper part of the annular outer ring is fixed It is fixedly connected to the bottom of the third support plate.

By adopting the above technical solution, the third support plate can be rotated during use. For some fat patients, it is difficult to sit on the third support without adjusting the direction of the third support plate The upper part of the board is arranged at the same time, so that the patient with a fat body can sit on the upper part of the third supporting board conveniently, thereby making the device more practical.

Further, a control switch is fixedly installed on the upper side of the second support plate, and the control switch is connected in series with the motor through a wire.

By adopting the above technical solution, it is convenient for medical personnel to control the operation of the motor by operating the control switch, and use the motor to drive the screw to run, so that the cylindrical support block moves up and down the screw under the restriction of the two guide rods, so that it can be easily adjusted The height of the third support plate can further adjust the position of the patient.

Further, the inside of the four second support rods are all hollow structures.

By adopting the above technical solution, the wire connecting the control switch and the motor is convenient to be routed through the inside of the second support rod, so that the appearance of the device is relatively simple.

Further, the bottom of the cylindrical support block is symmetrically provided with two guide holes respectively matched with the two guide rods, and the bottom of the cylindrical support block is located between the two guide holes A screw hole matched with the screw rod is also provided.

By adopting the above technical solution, the guide rod is convenient for restricting the rotation of the cylindrical support block, and the screw is convenient for driving the cylindrical support block to move up and down through thread transmission.

Further, the side surface of the cylindrical support block and the inner side surface of the cylindrical housing are disposed in close contact with each other.

By adopting the above technical solution, the cylindrical support block can be effectively prevented from tilting, and the screw can be prevented from being bent and damaged due to the tilt of the cylindrical support block.

Further, a silicone pad is also pasted on the upper side of the second support plate, and the color of the silicone pad is red.

By adopting the above technical solution, the silicone pad material is softer, which has a better cushioning effect on the patient's right arm, which can prevent the patient's right arm from directly contacting the upper surface of the second support plate, and avoid the upper surface of the second support plate The surface of the patient's right arm skin is scratched.

The color of the silicone pad is set to red, which makes the silicone pad more conspicuous, which can avoid the invisibility of older patients. At the same time, the red silicone pad also plays a role of identification, which can identify patients The area where the right arm is placed al lows the medical staff to measure the patient's blood pressure.

Further, a protective layer is also provided on the upper surface of the base, and the protective layer is prepared by the following method:

Weigh the following raw materials in parts by weight: 18-25 parts of epoxy resin, 8-12 parts of calcium carbonate powder, 10-12 parts of copper oxide powder, 12-16 parts of phenolic resin, 8-10 parts of quartz powder, polyurethane 15-20 parts, paraffin wax 3-5 parts, alcohol ester twelve 2-4 parts, triethanolamine 2-4 parts, emulsified silicone oil 1-3 parts and ethanol 30-50 parts;

S1. Add the weighed paraffin, alcohol ester twelve, triethanolamine, emulsified silicone oil and ethanol into the blender and stir for 20-30 min at a stirring speed of 600-700 r/min to prepare a mixed solution;

S2. Add epoxy resin, calcium carbonate powder, copper oxide powder, phenolic resin, quartz powder and polyurethane to the pulverizer for crushing, until the particle diameter of the material is not more than 40 um, and prepare a mixed powder material;

S3. Add the mixed solution prepared in step S1 and the mixed powder material prepared in step S2 to the reaction kettle for stirring for 20-30 min. The stirring speed of the reaction kettle is set at 700-900 r/min and the temperature is set at 60-70° C., to prepare protective coating;

S4. Polish the upper surface of the base with sandpaper, and then rinse it with a high-pressure water gun;

S5. Dry the base cleaned in step S4 with a hot air blower, and then use the high-pressure sprayer spray gun to spray the protective coating prepared in step S3 evenly on the upper surface of the dried base;

S6. Place the base 1 sprayed with protective paint in step S5 in a drying room for drying. The drying temperature is set at 80-100° C. and the time is set at 30-40 min, that is, a protective layer is prepared on the upper surface of the base.

By adopting the above technical scheme, the process steps for preparing protective coatings are simple and easy to realize. The prepared protective coatings have moderate viscosity, are not easy to delaminate, are easy to spray, generate no bubbles, and fully combine the components. After spraying, it can form a better coating film, which is not easy to produce cracks, and has a good film formation effect. The prepared protective layer has good anti-corrosion, abrasion resistance, anti-skid, anti-aging properties, good adhesion, not easy to fall off, can be effective Increase the anti-corrosion, wear-resistant, anti-skid, anti-aging performance of the base, so that the device has a long service life, and it is particularly important to prevent medical staff and patients from slipping when using the device.

In summary, the present invention mainly has the following beneficial effects:

1. In the present invention, the first support unit, the second support unit and the third support unit are all provided on the upper part of the base, and the integrated design is adopted, so that the structure of the entire device is relatively stable, which can effectively prevent the first support unit, the second support unit and the The falling phenomenon of the third support unit can avoid accidents caused by medical staff and patients who fall when measuring blood pressure, especially for hypertensive patients over 75 years old, accidental falls directly threaten their lives and safety, thus Effectively ensure the safety of patients during medical treatment;

2. In the present invention, the third support unit is driven by a motor to adjust the height of the third support plate, so when the patient sits on the upper part of the third support plate, the position of the patient can be adjusted by controlling the motor, and at the same time, the patient lays down the right arm In the upper part of the second support plate, the patient's right arm elbow and the patient's heart can be on the same level by adjusting the patient's position. At this time, the patient's heart position when measuring the blood pressure is completed, so that It makes the patient's blood pressure measurement result more accurate and the error is smaller, which is conducive to the patient's subsequent treatment, especially for the doctor to control the patient's medication;

3. According to the present invention, the third support plate can be rotated during use. For some fat patients, it is difficult to sit on the top of the third support plate without adjusting the direction of the third support plate. Therefore, the fatter patients can sit on the upper part of the third support plate conveniently, so that the device has better practicability;

4. In the present invention, the process steps for preparing protective coatings are simple and easy to realize. The prepared protective coatings have moderate viscosity, are not easy to delaminate, are easy to spray, generate no bubbles, and fully combine various components. The comprehensive performance is good, so that the protective coatings can be It forms a good coating film, is not easy to produce cracks, and has a good film formation effect. The prepared protective layer has good corrosion resistance, wear resistance, anti-skid, and anti-aging properties. It has good adhesion and is not easy to fall off. Anti-corrosion, abrasion resistance, anti-skid, anti-aging performance, so that the device has a long service life, it is particularly important to prevent medical staff and patients from slipping when using the device.

### FIGURE ILLUSTRATION

1 is a schematic structural diagram of an embodiment of the present invention;

FIG. 2 is one of the structural schematic diagrams from different perspectives of an embodiment of the present invention;

FIG. 3 is a second schematic structural view of an embodiment of the present invention from different perspectives;

4 is a schematic structural diagram of a first support unit according to an embodiment of the present invention;

5 is a schematic structural diagram of a second support unit according to an embodiment of the present invention;

6 is a schematic structural diagram of a third support unit according to an embodiment of the present invention;

7 is a schematic cross-sectional structural view of a third support unit according to an embodiment of the invention;

8 is a schematic diagram of an explosion structure of a rotating member according to an embodiment of the present invention;

9 is a schematic diagram of an explosion structure of a third support unit according to an embodiment of the present invention;

10 is a second schematic diagram of an explosion structure of a third support unit according to an embodiment of the present invention.

In the picture: 1. Base; 2. First support unit; 3. Second support unit; 4. Third support unit; 5. Rubber feet; 6. First support bar; 7. First support plate; 8. Two support rods; 9, second support plate; 10, control switch; 11, silicone pad; 12, cylindrical shell; 13, third support plate; 14, protective baffle; 15, motor; 16, coupling Device; 17, screw rod; 18, guide rod; 19, cylindrical support block; 20, circular plate; 21, limit ring; 22, annular outer ring; 23, threaded hole; 24, guide hole; 26, annular inner ring; 27, rotating parts.

### SPECIFIC IMPLEMENTATION MODALITIES

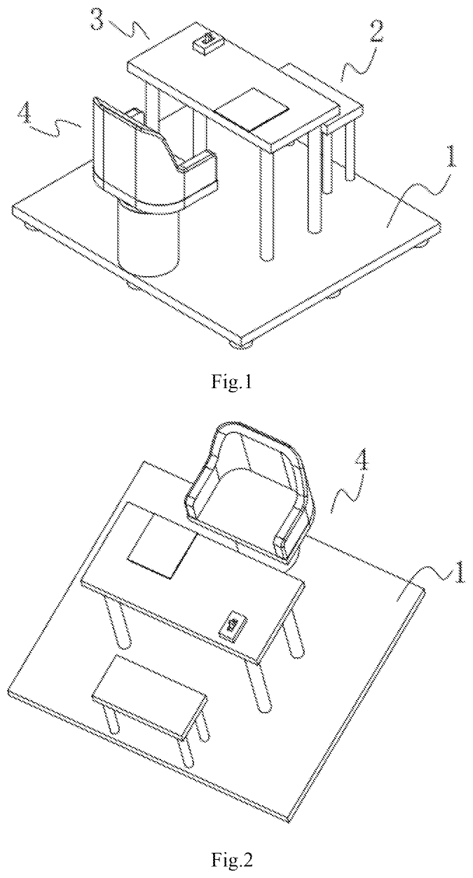

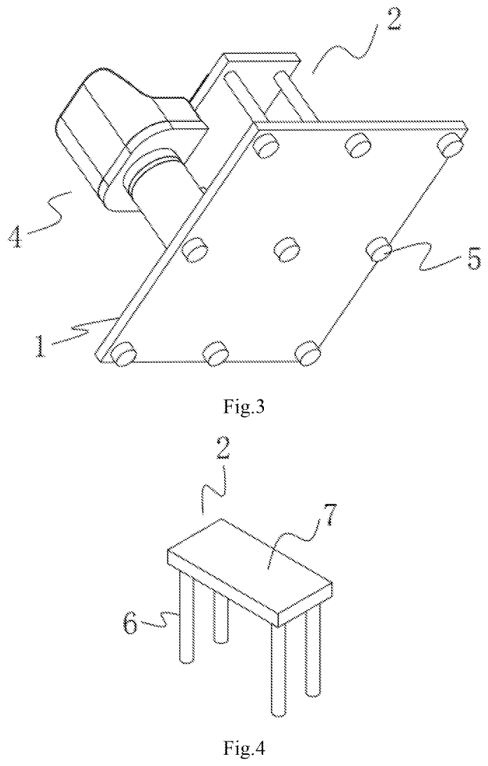

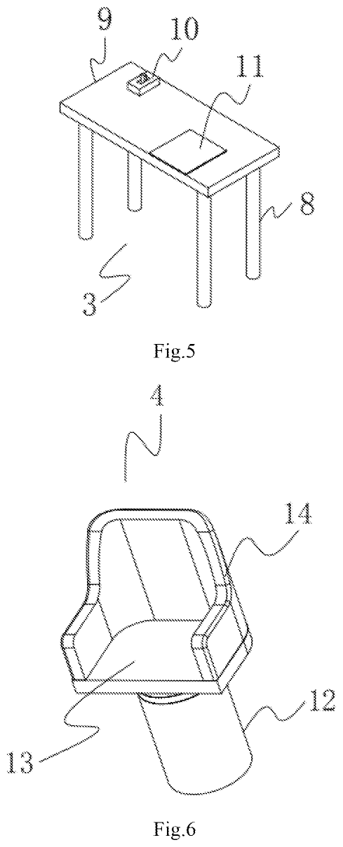

The present invention will be further described in detail below with reference to FIGS. 1-10.

### Example 1

A device for correcting the position of the heart when measuring blood pressure, as shown in FIGS. Unit 4;

As shown in FIG. 4, the first support unit 2 includes four first support rods 6 and a first support plate 7. The four first support rods 6 are symmetrically and vertically mounted on the upper portion of the base 1. The first support plate 7 is fixedly installed on the upper ends of the four first support rods 6;

As shown in FIG. 5, the second support unit 3 includes four second support rods 8 and a second support plate 9. The four second support rods 8 are symmetrically and vertically mounted on the upper portion of the base 1. The second support plate 9 is fixedly installed on the upper ends of the four second support rods 8;

As shown in FIGS. 6-7 and 9-10, the third support unit 4 includes a cylindrical casing 12 fixedly mounted on the upper portion of the base 1, the upper end of the cylindrical casing 12 is an open structure, Moreover, a motor 15 is fixedly installed in the upper part of the inner bottom wall of the cylindrical casing 12, and a rotating shaft of the motor 15 is fixedly installed with a screw 17 through a coupling 16. The inside of the cylindrical casing 12 A cylindrical support block 19 is also slideably mounted above the motor 15, and the upper part of the inner bottom wall of the cylindrical housing 12 is symmetrically fixed around the motor 15 and two vertically arranged guide rods are symmetrically fixed 18, the upper ends of the two guide rods 18 slide into the bottom of the cylindrical support block 19, the upper end of the screw 17 is screwed into the bottom of the cylindrical support block 19, the cylinder A circular plate 20 is fixedly installed on the upper portion of the type supporting block 19, the circular plate 20 is located above the cylindrical housing 12, and a third supporting plate 13 is fixedly installed on the upper portion of the circular plate 20 through a rotating member 27.

The first support unit 2 is used as a seat for medical staff when diagnosing patients; the second support unit 3 is used as an examination table when measuring blood pressure; the third support unit 4 is used as a seat for patients when measuring blood pressure; The supporting unit 2, the second supporting unit 3 and the third supporting unit 4 are all arranged on the upper part of the base 1, and adopt an integrated design, so that the structure of the whole device is relatively stable, which can effectively prevent the first supporting unit 2, the second supporting unit 3 and the The falling phenomenon of the third support unit 4 can avoid the accident of falls of medical staff and patients when measuring blood pressure, especially for hypertensive patients aged over 75 years, accidental falls directly threaten their lives and safety. In addition, the third support unit 4 is driven by the motor 15 to adjust the height of the third support plate 13, so when the patient sits on the upper portion of the third support plate 13, the position of the patient can be adjusted by controlling the motor 15 and the patient will When the right arm is placed flat on the upper part of the second support plate 9, the patient's right arm elbow and the patient's heart can be on the same level by adjusting the position of the patient. Proofread the position, so that the patient's blood pressure measurement results are more accurate and the error is smaller, which is conducive to the patient's subsequent treatment, especially for the doctor to control the patient's medication.

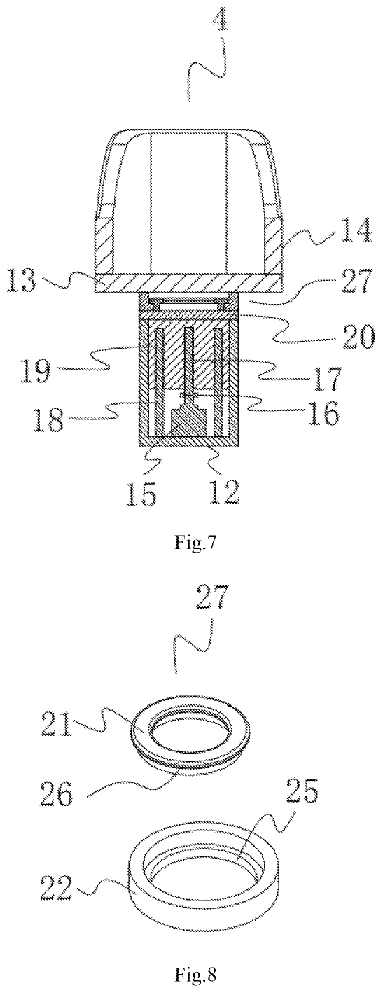

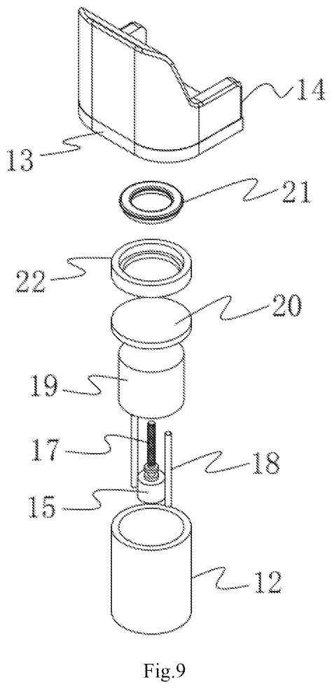

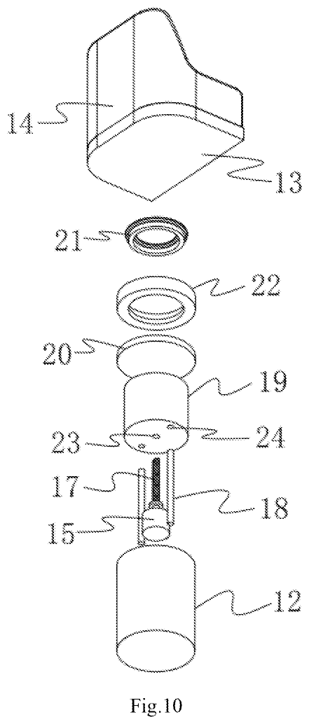

Preferably, as shown in FIG. 3, a plurality of rubber feet 5 are fixedly installed on the bottom of the base 1.

When the device is installed, a number of rubber feet 5 have a better support for the entire device, at the same time, it can prevent the base 1 from scratching the support surface of the device and increase the static friction between the base 1 and the support surface of the device, Can prevent the device from slipping during use.

Preferably, as shown in FIGS. 1-3 and 6-7 and 9-10, a protective baffle 14 is also fixedly installed on the upper portion of the third support plate 13.

The protective baffle 14 is used to protect the patient when the patient is sitting on the upper portion of the third support plate 13, which can prevent the patient from accidentally falling from the upper portion of the third support plate 13, thereby making the device safe and reliable.

Preferably, the rotating member 27 includes an annular outer ring 22 and an annular inner ring 26, an inner bottom of the annular outer ring 22 is integrally provided with a retaining ring 25, and an upper portion of the annular inner ring 26 is integrally provided with a limit ring 21, The annular inner ring 26 is movably snapped inside the annular outer ring 22 through the cooperation of the limit ring 21 and the retaining ring 25, and the bottom of the annular inner ring 26 is fixed to the circular plate The upper part of 20 is fixedly connected, and the upper part of the annular outer ring 22 is fixedly connected with the bottom of the third support plate 13.

The third support plate 13 can be rotated during use. For some fat patients, it is difficult to sit on the top of the third support plate 13 without adjusting the direction of the third support plate At the same time, the above settings make it easier for patients with a fat body to sit on the upper part of the third support plate 13, thereby making the device more practical.

Preferably, as shown in FIGS. 1-2 and 5, a control switch 10 is fixedly installed on the upper side of the second support plate 9, and the control switch 10 is connected in series with the motor 15 through a wire.

It is convenient for medical personnel to control the operation of the motor 15 by operating the control switch 10, and use the motor 15 to drive the screw 17 to run, so that the cylindrical support block 19 moves up and down along the screw 17 under the restriction of the two guide rods 18, which can be conveniently Adjust the height of the third support plate 13 to adjust the position of the patient.

Preferably, the insides of the four second support rods 8 are all hollow structures.

The wire that facilitates the connection between the control switch 10 and the motor 15 is routed through the inside of the second support rod 8, so that the appearance of the device is relatively simple.

Preferably, as shown in FIG. 10, the bottom of the cylindrical support block 19 is symmetrically provided with two guide holes 24 respectively matched with the two guide rods 18, and the cylindrical support block 19 has A screw hole 23 matching the screw rod 17 is also provided inside the bottom between the two guide holes 24.

The guide rod 18 is convenient for restricting the rotation of the cylindrical support block 19, and the screw 17 is convenient for driving the cylindrical support block 19 to move up and down through thread transmission.

Preferably, as shown in FIG. 7, the side surface of the cylindrical support block 19 and the inner side surface of the cylindrical housing 12 are closely arranged.

The phenomenon that the cylindrical support block 19 is tilted can be effectively prevented, and the screw 17 can be prevented from being bent and damaged due to the tilt of the cylindrical support block 19.

Preferably, as shown in FIGS. 1-2 and 5, the upper side of the second support plate 9 is also pasted with a silicone pad 11, the color of the silicone pad 11 is red.

The material of the silicone pad 11 is relatively soft, and it has a good cushioning effect on the patient's right arm, which can prevent the patient's right arm from directly contacting the upper surface of the second support plate 9 and prevent the upper surface of the second support plate 9 from being injured On the skin of the patient's right arm, the color of the silicone pad 11 is set to red, which makes the silicone pad 11 more conspicuous, which can avoid the invisibility of older patients. At the same time, the red silicone pad 11 also plays a role of identification, which can identify patients The area where the right arm is placed al lows the medical staff to measure the patient's blood pressure.

The motor 15 described in this embodiment can be a fixed-speed motor model STS 25W-200W produced by Taiwan Chenggang Motor (Shanghai Taicang Kunshan) Co., Ltd. In addition, the upper adjustment of the cylindrical support block 19 in this embodiment The posit ion can be higher than the upper surface of the second support plate 9, in actual use, the upper part of the cylindrical support block 19 will not be adjusted higher than the upper surface of the second support plate 9, so there is no need to worry about the cylindrical support block 19 The screw 17 is disengaged. In addition, a stroke switch can be added to cooperate with the motor 15 to prevent the cylindrical support block 19 from disengaging from the screw 17. The technical means for adding the stroke switch is the existing technology, and here, no more Repeat.

### Example 2

The difference from Embodiment 1 is that the upper surface of the base 1 is further provided with a protective layer, and the protective layer is prepared by the following method:

Weigh the following raw materials in parts by weight: 18 parts of epoxy resin, 8 parts of calcium carbonate powder, 10 parts of copper oxide powder, 12 parts of phenolic resin, 8 parts of quartz powder, 15 parts of polyurethane, 3 parts of paraffin, 12 parts of alcohol ester Parts, 2 parts of triethanolamine, 1 part of emulsified silicone oil and 30 parts of ethanol;

S1, add the weighed paraffin, alcohol ester twelve, triethanolamine, emulsified silicone oil and ethanol into the blender and stir for 20 min, the stirring speed is 600 r/min to prepare a mixed solution;

S2. Add epoxy resin, calcium carbonate powder, copper oxide powder, phenolic resin, quartz powder and polyurethane to the pulverizer for crushing, until the particle diameter of the material is not more than 40 um, and prepare a mixed powder material;

S3. The mixed solution prepared in step S1 and the mixed powder material prepared in step S2 are added to the reaction kettle and stirred for 20 minutes. The stirring speed of the reaction kettle is set to 700 r/min and the temperature is set to 60° C. Protective coatings;

S4. Polish the upper surface of the base 1 with sandpaper, and then rinse it with a high-pressure water gun;

S5. Dry the base 1 cleaned in step S4 with a hot air blower, and then use the high-pressure sprayer spray gun to spray the protective coating prepared in step S3 evenly on the upper surface of the base 1 after drying;

S6. Place the base 1 sprayed with protective paint in step S5 in a drying room for drying. The drying temperature is set to 80° C. and the time is set to 30 min. That is, a protective layer is prepared on the upper surface of the base 1.

### Example 3

The difference from Example 2 is the preparation of the protective layer. The specific preparation method is as follows:

Weigh the following raw materials in parts by weight: 23 parts of epoxy resin, 10 parts of calcium carbonate powder, 11 parts of copper oxide powder, 14 parts of phenolic resin, 9 parts of quartz powder, 18 parts of polyurethane, 4 parts of paraffin, 12 parts of alcohol ester Parts, 3 parts of triethanolamine, 2 parts of emulsified silicone oil and 40 parts of ethanol;

S1. Add the weighed paraffin wax, alcohol ester twelve, triethanolamine, emulsified silicone oil and ethanol into the blender and stir for 25 min at a stirring speed of 650 r/min to prepare a mixed solution;

S2. Add epoxy resin, calcium carbonate powder, copper oxide powder, phenolic resin, quartz powder and polyurethane to the pulverizer for crushing, until the particle diameter of the material is not more than 40 um, and prepare a mixed powder material;

S3. Add the mixed solution prepared in step S1 and the mixed powder material prepared in step S2 to the reaction kettle and stir for 25 min. The stirring speed of the reaction kettle is set to 800 r/min and the temperature is set to 65° C. Protective coatings;

S4. Polish the upper surface of the base 1 with sandpaper, and then rinse it with a high-pressure water gun;

S5. Dry the base 1 cleaned in step S4 with a hot air blower, and then use the high-pressure sprayer spray gun to spray the protective coating prepared in step S3 evenly on the upper surface of the base 1 after drying;

S6. Place the base 1 sprayed with protective paint in step S5 in a drying room for drying, the drying temperature is set to 90° C., and the time is set to 35 min, that is, a protective layer is prepared on the upper surface of the base 1.

### Example 4

The difference from Example 2 is the preparation of the protective layer. The specific preparation method is as follows:

Weigh the following raw materials in parts by weight: 25 parts of epoxy resin, 12 parts of calcium carbonate powder, 12 parts of copper oxide powder, 16 parts of phenol resin, 10 parts of quartz powder, 20 parts of polyurethane, 5 parts of paraffin, twelve alcohol esters 4 Parts, 4 parts of triethanolamine, 3 parts of emulsified silicone oil and 50 parts of ethanol;

S1. Add the weighed paraffin wax, alcohol ester twelve, triethanolamine, emulsified silicone oil and ethanol into the blender and stir for 30 min at a stirring speed of 700 r/min to prepare a mixed solution;

S2. Add epoxy resin, calcium carbonate powder, copper oxide powder, phenolic resin, quartz powder and polyurethane to the pulverizer for crushing, until the particle diameter of the material is not more than 40 um, and prepare a mixed powder material;

S3. Add the mixed solution prepared in step S1 and the mixed powder material prepared in step S2 to the reaction kettle and stir for 30 min. The stirring speed of the reaction kettle is set to 900 r/min and the temperature is set to 70° C. Protective coatings;

S4. Polish the upper surface of the base 1 with sandpaper, and then rinse it with a high-pressure water gun;

S5. Dry the base 1 cleaned in step S4 with a hot air blower, and then use the high-pressure sprayer spray gun to spray the protective coating prepared in step S3 evenly on the upper surface of the base 1 after drying;

S6. Place the base 1 sprayed with protective paint in step S5 in a drying room for drying, the drying temperature is set to 100° C., and the time is set to 40 min, that is, a protective layer is prepared on the upper surface of the base 1.

The comparison results of the static friction coefficient of the upper surface of the base 1 in Example 1-4 in the laboratory under the same conditions using a dynamic and static friction coefficient tester are as follows:

| | Coefficient of dynamic |
|---|---|
| | and static friction |
| Example 1 | 0.35 |
| Example 2 | 0.65 |
| Example 3 | 0.68 |
| Example 4 | 0.67 |

From the comparative analysis of the test results in the above table, it can be seen that Example 3 is the best example. By adopting the above technical solution, the process steps for preparing protective coatings are simple and easy to implement. The prepared protective coatings have moderate viscosity, are not easy to delaminate, are easy to spray, and have no bubbles Produced and fully combined with various components, the overall performance is good, so that the protective coating can form a better coating film after spraying, it is not easy to produce cracks, and the film formation effect is better. The prepared protective layer has good corrosion resistance, wear resistance, Anti-skid, anti-aging performance, good adhesion, not easy to fall off, can effectively increase the anti-corrosion, wear-resistant, anti-skid, anti-aging performance of the base 1, so that the device has a longer service life, especially important is to prevent medical personnel The patient slipped when using the device with the patient.

Working principle: In the device for checking the position of the heart when measuring blood pressure, the first support unit 2 is used as a seat for medical staff to diagnose patients; the second support unit 3 is used as an examination table when measuring blood pressure; and the third support unit 4 It is used as a seat for patients to measure blood pressure; this device sets the first support unit 2, the second support unit 3 and the third support unit 4 all on the upper part of the base 1, adopts an integrated design, so that the whole device structure is relatively stable, It can effectively prevent the first support unit 2, the second support unit 3 and the third support unit 4 from falling down, and can avoid the accidents of medical staff and patients falling when measuring blood pressure, especially for those over 75 years old For hypertensive patients, accidental falls directly threaten their life safety. In addition, the third support unit 4 is driven by a motor 15 to adjust the height of the third support plate 13, so when the patient sits on the upper part of the third support plate 13, The position of the patient can be adjusted by controlling the motor 15 and the patient's right arm is placed on the upper part of the second support plate 9 at the same time. By adjusting the position of the patient, the elbow of the patient's right arm and the heart of the patient can be On the same level, the patient's heart position when blood pressure is measured is now corrected, so that the patient's blood pressure measurement result is more accurate and the error is smaller, which is conducive to the patient's subsequent treatment, especially for the doctor to control the patient's medication.

Instructions:

The first step, when using for the first time, first wipe the entire device with a clean cloth dipped in alcohol;

In the second step, the sphygmomanometer is then ready to be placed on the upper part of the second support plate 9;

The third step is to energize the device and let the patient sit on the upper part of the third support plate 13. If you encounter a fat patient, you can rotate the third support plate 13 to the side and let the patient sit on the third support The upper part of the plate 13 and allow the patient to rotate the third support plate 13 so that the patient is facing the second support plate 9;

The fourth step, the medical staff sits on the upper part of the first support plate 7 and al lows the patient to take off the clothes of the right arm, preferably to expose the skin, and then the patient places the front end of the right arm on the upper part of the second support plate 9, The medical staff controls the operation of the motor 15 by operating the control switch 10, and uses the motor 15 to drive the screw 17 to run, so that the cylindrical support block 19 moves up and down along the screw 17 under the restriction of the two guide rods 18, which can be easily adjusted The height of the third support plate 13 makes the elbow of the right arm of the patient and the heart of the patient on the same level. At this time, the position of the heart of the patient when measuring the blood pressure is completed. Blood pressure is measured.

Installation Method:

The first step is to install a number of rubber feet 5 evenly on the bottom of the base 1;

The second step is to install the first support unit 2, weld the four first support rods 6 to the upper part of the base 1, and then weld the first support plate 7 to the upper ends of the four first support rods 6;

The third step is to install the second support unit 3, weld the four second support bars 8 to the upper part of the base 1, and then weld the second support plate 9 to the upper ends of the four second support bars 8;

The fourth step is to install the third support unit 4, weld the cylindrical housing 12 to the upper part of the base 1, and then install the motor 15 and two guide rods 18, the motor 15 can be installed with bolts, the two guide rods 18 can be used Welding method, and then the rotating shaft of the motor 15 is fixedly connected to one end of the screw 17 through the coupling 16. After the motor is installed, the control switch 10 can be installed and wired. To facilitate wiring, it can be opened on the base 1 There is a wiring slot, and then align the guide holes 24 in the bottom of the cylindrical support block 19 with the upper ends of the two guide rods 18, and then operate the control switch 10 to turn on the motor 15 to drive the screw rod 17 to rotate, and drill the screw rod 17 Into the threaded hole 23 inside the bottom of the cylindrical support block 19, at this time the cylindrical support block 19 will enter the interior of the cylindrical housing 12, and then the circular plate 20 is bolted to the upper part of the cylindrical support block 19, The rotating member 27 is welded to the upper portion of the circular plate 20, the third supporting plate 13 is welded to the upper portion of the rotating member 27, and the protective baffle 14 is fixedly installed on the upper portion of the third supporting plate 13, that is, the installation is completed.

The parts not involved in the present invent ion are the same as the prior art or can be implemented by using the prior art.

This specific embodiment is merely an explanation of the present invention, and it is not a limitation of the present invention. Those skilled in the art can make modifications without creative contribution to this embodiment after reading this specification, as long as they have rights in the present invention Within the scope of the requirements are protected by patent law.

1. A device for correcting the position of the heart when measuring blood pressure, including a base (1), characterized in that a first support unit (2) and a second support unit (3) are sequentially arranged on the upper part of the base (1) And the third support unit (4);
The first support unit (2) includes four first support rods (6) and a first support plate (7), and the four first support rods (6) are symmetrically and vertically mounted on the base (1), The first support plate (7) is fixedly installed on the upper ends of the four first support rods (6);
The second support unit (3) includes four second support rods (8) and a second support plate (9), and the four second support rods (8) are symmetrically and vertically mounted on the base (1), The second support plate (9) is fixedly installed on the upper ends of the four second support rods (8);
The third support unit (4) includes a cylindrical casing (12) fixedly mounted on the upper portion of the base (1), the upper end of the cylindrical casing (12) is an open structure, and the circle A motor (15) is fixedly mounted on the upper part of the inner bottom wall of the cylindrical casing (12), and a rotating shaft of the motor (15) is fixedly installed with a screw (17) through a coupling (16), and the cylinder A cylindrical support block (19) is slidably installed inside the casing (12) above the motor (15), and the upper part of the inner bottom wall of the cylindrical casing (12) is located on the motor (15) Two vertically arranged guide rods (18) are symmetrically fixedly installed around, and the upper ends of the two guide rods (18) slide into the bottom of the cylindrical support block (19), the The upper end of the screw rod (17) is screwed inside the bottom of the cylindrical support block (19), a circular plate (20) is fixedly installed on the upper part of the cylindrical support block (19), and the circular plate (20) A third support plate (13) is fixedly installed on the upper portion of the circular plate (20) through a rotating member (27).

2. The device for correcting the position of the heart when measuring blood pressure according to claim 1, characterized in that a plurality of rubber feet (5) are fixedly installed on the bottom of the base (1).

3. The device for correcting the position of the heart when measuring blood pressure according to claim 1, characterized in that a protective baffle (14) is also fixedly installed on the upper part of the third support plate (13).

4. The device for correcting the position of the heart when measuring blood pressure according to claim 1, characterized in that the rotating member (27) includes an annular outer ring (22) and an annular inner ring (26), the annular outer The inner bottom of the ring (22) is integrally provided with a retaining ring (25), the upper part of the annular inner ring (26) is integrally provided with a limit ring (21), the annular inner ring (26) passes through the limit ring (21) The movable ring is engaged with the retaining ring (25) inside the annular outer ring (22), and the bottom of the annular inner ring (26) is fixed to the upper part of the circular plate (20) Connected, the upper part of the annular outer ring (22) is fixedly connected with the bottom of the third support plate (13).

5. The device for correcting the position of the heart when measuring blood pressure according to claim 1, characterized in that a control switch (10) is fixedly installed on the upper side of the second support plate (9) (10) The motor (15) is connected in series through a wire.

6. The device for correcting the position of the heart when measuring blood pressure according to claim 1, characterized in that the inside of the four second support rods (8) are all hollow structures.

7. The device for correcting the position of the heart when measuring blood pressure according to claim 1, wherein the bottom of the cylindrical support block (19) is symmetrically provided with two and two guide bars 18) Matching guide holes (24), and the bottom of the cylindrical support block (19) is located between the two guide holes (24) and is matched with the screw (17) Threaded hole (23).

8. The device for correcting the position of the heart when measuring blood pressure according to claim 1, characterized in that the side of the cylindrical support block (19) is in contact with the inner side of the cylindrical housing (12) settings.

9. The device for correcting the position of the heart when measuring blood pressure according to claim 1, characterized in that a silicone pad (11) is adhered to the upper side of the second support plate (9), the silicone The color of the pad (11) is red.

10. The device for correcting the position of the heart when measuring blood pressure according to claim 1, characterized in that a protective layer is further provided on the upper surface of the base (1), and the protective layer is prepared by the following method:
Weigh the following raw materials in parts by weight: 18-25 parts of epoxy resin, 8-12 parts of calcium carbonate powder, 10-12 parts of copper oxide powder, 12-16 parts of phenolic resin, 8-10 parts of quartz powder, polyurethane 15-20 parts, paraffin wax 3-5 parts, alcohol ester twelve 2-4 parts, triethanolamine 2-4 parts, emulsified silicone oil 1-3 parts and ethanol 30-50 parts;
S1. Add the weighed paraffin, alcohol ester twelve, triethanolamine, emulsified silicone oil and ethanol into the blender and stir for 20-30 min at a stirring speed of 600-700 r/min to prepare a mixed solution;
S2. Add epoxy resin, calcium carbonate powder, copper oxide powder, phenolic resin, quartz powder and polyurethane to the pulverizer for crushing, until the particle diameter of the material is not more than 40 um, and prepare a mixed powder material;
S3. Add the mixed solution prepared in step S1 and the mixed powder material prepared in step S2 to the reaction kettle for stirring for 20-30 min. The stirring speed of the reaction kettle is set at 700-900 r/min and the temperature is set at 60-70° C., to prepare protective coating;
S4. Polish the upper surface of the base (1) with sandpaper, and then rinse it with a high-pressure water gun;
S5. Dry the base (1) cleaned in step S4 with a hot air blower, and then spray the protective coating prepared in step S3 on the upper surface of the dried base (1) evenly using a high-pressure spray gun;
S6. Place the base (1) sprayed with protective paint in step S5 in the drying room for drying, the drying temperature is set to 80-100° C., the time is set to 30-40 min, that is, the protection is made on the upper surface of the base (1) Floor.
